(19) United States
(12) Patent Application Publication
(10) Pub. No.: US 2006/0058331 A1
(43) Pub. Date: Mar. 16, 2006

(54) Abuse resistant opioid dosage form

(76) Inventors: Bradley Galer, West Chester, PA (US); Huaihung D Kao, Syosset, NY (US); Michelle Howard Sparks, Ridgewood, NY (US); Yadi Zeng, Fort Lee, NJ (US)

Correspondence Address: DILWORTH & BARRESE, LLP, 333 EARLE OVINGTON BLVD., UNIONDALE, NY, 11553, US

(21) Appl. No.: 10/514,390

(22) PCT Filed: May 13, 2003
(86) PCT No.: PCT/US03/14839

§ 371 Date: Jul. 7, 2005

Related U.S. Application Data
(60) Provisional application No. 60/453,699, filed on May 13, 2002.

(51) Int. Cl. A61K 31/485

(52) U.S. Cl. 514/282

(57) ABSTRACT

The present invention pertains to a pharmaceutical dosage form comprising an opioid analgesic and a nontoxic N-methyl-D-aspartate receptor antagonist wherein the pharmaceutical dosage form is substantially free of an opioid antagonist The nontoxic N-methyl-D-aspartate receptor antagonist is present in an opioid euphoria-inhibiting amount to prevent or discourage abuse.

### CROSS-REFERENCE TO RELATED APPLICATIONS

This application claims the benefit under 35 U.S.C. § 119(e) of earlier filed and copending U.S. Provisional Application No. 60/453,699, filed May 13, 2002, the contents of which are incorporated by reference herein.

### BACKGROUND OF THE INVENTION

1. Field of the Invention

The present invention relates to an abuse-resistant opioid-containing pharmaceutical dosage form having an analgesically effective amount of an opioid and an opioid euphoria-inhibiting amount of at least one nontoxic N-methyl-D-aspartate receptor antagonist, where the dosage form is substantially free of opioid antagonist.

2. Description of the Related Art

Morphine, a classic opioid, has been known as a very powerful analgesic compound for many years. Its potential as a target of abuse has been known for almost as long. Opioids and their derivatives are used in the pharmaceutical industry as narcotic analgesics, hypnotics, sedatives, anti-diarrheals, anti-spasmotics, and antitussives. Despite their well known potential for addiction and abuse, opioids are widely used due to their superior, powerful analgesic properties.

In the past, abuse of opioids was generally limited to illicit drugs made in illegal laboratories. Abuse of pharmaceutical opioids was quite limited. Accordingly, action by makers of pharmaceutical opioids would, in the past, have little or no effect on illegal abuse of opioids.

Recently, however, this trend has been changing and abuse of pharmaceutical opioids has been increasing. This is especially true in the case of extended release opioid dosage forms. One reason for the increase of abuse is that extended release opioid dosage forms are intended for decreased frequency of dosing, which results in the production of dosage forms having substantially increased amounts of opioid. Therefore, an extended release dosage form can provide much more opioid to the potential abuser than the past low dose, immediate release dosage forms.

Two examples of previous attempts to curtail abuse, U.S. Pat. Nos. 6,228,863 and 6,277,384, both disclose single unit dosage forms containing an opioid, an opioid antagonist and, optionally, any of a third group of drugs among which are mentioned NMDA receptor antagonists. The opioid antagonist counteracts the euphoric effects of the opioid and renders the dosage form less likely to be abused. However, there is no mention in either of these patents of the ability of an NMDA receptor antagonist to inhibit the euphoria-inducing properties of an opioid analgesic.

N-methyl-D-aspartate (NMDA) receptor antagonists are well known in the art and encompass, for example, dextromethorphan, dextrorphan, memantine, amantidine, d-methadone and their pharmaceutically acceptable salts. NMDA receptor antagonists are known to inhibit the development of tolerance to and/or dependence on addictive drugs, e.g., narcotic analgesics such as morphine, codeine, etc., as disclosed in U.S. Pat. Nos. 5,321,012 and 5,556,838, and to treat chronic pain as disclosed in U.S. Pat. No. 5,502,058, the contents of each of which are incorporated by reference herein.

Controlled release dosage forms for pharmaceuticals, which include extended release and sustained release dosage forms, are known to those skilled in the art. See, e.g., U.S. Pat. Nos. 4,861,598, 4,970,075, 5,266,331, 5,508,042, 5,549,912, 5,656,295, 5,958,459, 5,968,551, 6,103,261, 6,143,322, 6,143,353, and 6,294,195, the contents of each of which are incorporated by reference herein. For example, U.S. Pat. Nos. 4,861,598 and 4,970,075 disclose controlled release pharmaceutical compositions for oral administration having extended action due to their use of a higher aliphatic alcohol and acrylic resin as their base material. Pharmaceutically active agents utilized with these compositions include narcotics. U.S. Pat. Nos. 5,266,331, 5,508,042, 5,549,912 and 5,656,295 disclose solid controlled release oral dosage forms of oxycodone or its salts whereby the oxycodone is encompassed in a carrier with a defined dissolution rate for the extended release of the pharmaceutical in vitro.

With the increase in the abuse of extended release opioid compositions, it would be beneficial to develop a dosage form which has the benefits of the opioid analgesics but reduces their euphoric effects in those dependent on opioids as well as the general population.

### BRIEF SUMMARY OF THE INVENTION

The present invention relates to an abuse-resistant opioid-containing pharmaceutical dosage form comprising an analgesically effective amount of an opioid analgesic and an opioid euphoria-inhibiting amount of at least one nontoxic N-methyl-D-aspartate antagonist, whereby the dosage form is substantially free of opioid antagonist.

The nontoxic N-methyl-D-aspartate antagonist will, because of its dysphoric effects, inhibit or discourage abuse of the dosage form. In addition, when administered intranasally, the nontoxic N-methyl-D-aspartate antagonist will act as an irritant to the nasal mucosa and thus inhibit or discourage abuse of the dosage form via intranasal administration.

With oral and nasal abuse, abusers chew or crush a controlled release opioid tablet to convert the tablet to immediate release. Abusers then take the crushed tablet orally or intranasally (by snorting the powder) in order to obtain a euphoria or high. Thus, the pharmaceutical dosage form of the present invention will discourage nasal and oral abuse of orally administered controlled release solid dosage forms which in recent years have become much more widely abused.

If the pharmaceutical dosage form is dissolved and injected, the NMDA receptor antagonist will prevent the abuser from receiving a euphoric high. This is due both to the increased efficacy of the antagonist when injected, as well as to the high doses of antagonist released by the crushed pharmaceutical dosage form. Thus, the pharmaceutical dosage form of the present invention will prevent abuse by administration of the dosage when injected.

### DETAILED DESCRIPTION OF THE INVENTION

The present invention is directed to pharmaceutical dosage forms comprising a combination of an analgesically effective amount of an opioid analgesic and a euphoria-inhibiting amount of a nontoxic opioid euphoria-inhibiting NMDA receptor antagonist, where the dosage form is substantially free of opioid antagonists. Opioid antagonists are undesirable because they can precipitate withdrawal when taken by a chronic opioid abuser.

The nontoxic NMDA receptor antagonist is present in an amount (i) which does not cause a reduction in the level of analgesia elicited from the dosage form to a non-therapeutic level and (ii) which provides at least a mildly negative, “aversive” experience in physically dependent subjects when the subjects attempt to take at least 2 times the usually prescribed dose at a time (and often 5-10 times that dose or more), as compared to a comparable dose of the opioid without the NMDA receptor antagonist present.

The first component of the abuse-resistant opioid-containing pharmaceutical dosage form is an analgesically effective amount of an opioid analgesic. Opioid analgesics suitable for use in the solid dosage form generally have a potential for abuse and include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

The preferred dosage of opioid analgesic can range from about 1 mg per 70 kg body weight to about 800 mg per 70 kg body weight per unit dose. Preferably, the dosage of opioid analgesic is from about 10 mg per 70 kg body weight to about 500 mg per 70 kg body weight in the unit dosage form. Where the opioid analgesic is fentanyl or sufentanyl, the preferred dosage is from about 5 μg per 70 kg to about 250 kg per 70 kg body weight per unit dose.

The second component of the abuse-resistant opioid-containing pharmaceutical solid dosage form is an opioid euphoria-inhibiting amount of at least one nontoxic opioid euphoria-inhibiting NMDA receptor antagonist. Nontoxic opioid euphoria-inhibiting NMDA receptor antagonists suitable for use in accordance with the present invention include dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4,4-diphenyl-3-heptanone hydrochloride), their mixtures and their pharmaceutically acceptable salts. Dextromethorphan is a preferred NMDA receptor antagonist for use herein due to its ready availability and wide acceptance as an ingredient of many over-the-counter medications where it is utilized for its cough-suppressant (antitussive) activity. Not only will the dextromethorphan inhibit the euphoria-producing effects of the opioid but, when the dosage form is abused intranasally, it will also act as an irritant to the nasal mucosa and thus deter or inhibit abuse of the opioid by intranasal administration.

The term “nontoxic” as used herein shall be understood in a relative sense and is intended to designate any substance that has been approved by the United States Food and Drug Administration (“FDA”) for administration to humans or, in keeping with established regulatory criteria and practice, is susceptible to approval by the FDA for administration to humans. The term “nontoxic” is also used herein to distinguish the NMDA receptor antagonists that are useful in the practice of the present invention from NMDA receptor antagonists such as MK 801 (the compound 5-methyl-10,11-dihydro-SH-dibenze[a,d]cyclohepten-5,10-imine), CPP (the compound 3-[2-carboxypiperazin-4-yl]propyl-1-phosphonic acid) and PCP (the compound 1-(1-phenylcyclohexyl)piperidine) whose toxicities effectively preclude their therapeutic use.

For purposes of this disclosure, the expression “opioid euphoria-inhibiting” includes the suppression, cloaling, masking or countering of the euphoria-inducing properties of opioids, e.g., by a mechanism of dysphoria, but excludes any mechanism involving opioid antagonism. It will be appreciated by one skilled in the art that the NMDA receptor antagonists discussed in U.S. Pat. No. 5,321,012 are not necessarily “opioid euphoria-inhibiting” amounts. In accordance with the present disclosure, opioid antagonists are undesirable since they pose a risk of precipitating opioid withdrawal when taken by a chronic opioid abuser.

For purposes of this disclosure, “controlled release” includes “extended release” and “sustained release” and pertains to the release of pharmaceutical agents at a defined level over an extended period of time.

The expression “dosage form” is understood to include “unit dosage form”. The expression “unit dosage form” means a physically discrete unit which contains specified amounts of the opioid analgesic and nontoxic NMDA receptor antagonist, in combination with a carrier and/or any other pharmacologically active substance or pharmaceutical excipient, which amounts are selected so that a fixed number, e.g. one, of the units is suitable to achieve a desired therapeutic effect.

All modes of administration are contemplated, e.g., orally, rectally, parenterally, intrathecally, intranasally, transdermally, and topically.

Additionally, the pharmaceutical dosage form herein can optionally contain at least one other pharmacologically active substance e.g., an analgesically useful amount of a non-narcotic analgesic such as acetaminophen, nonsteroidal anti-inflammatory drug (NSAID) such as aspirin, bromfenac, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and the like, cyclooxygenase-II (COX II) inhibitor such as celecoxib (Celebrex), rofecoxib (Vioxx), meloxicam, L-745337 (Merck), MK-966 (Merck), L-768277 (Merck), GR-253035 (Glaxo-Wellcome), JTE-S22 (Japan Tobacco), RS-57067-000 (Roche), SC-58125 (Searle), SC-078 (Searle), PD-138387 (Warner-Lambert), NS-398 (Taisho), flosulide and PD-164387 (Warner-Lambert), or other COX-II inhibitor such as any of those described in, e.g., U.S. Pat. Nos. 5,616,601; 5,604,260; 5,593,994; 5,550,142; 5,536,752; 5,521,213; 5,474,995; 5,639,780; 5,604,253; 5,552,422; 5,510,368; 5,436,265; 5,409,944; and 5,130,311, all of which are hereby incorporated by reference.

The preferred dosage of nontoxic NMDA receptor antagonist can range from about 100 mg per 70 kg body weight to about 500 mg per 70 kg body weight per unit dose. Preferably, the dosage of nontoxic NMDA receptor antagonist is from about 200 mg per 70 kg body weight to about 400 mg per 70 kg body weight, with a range of about 225 mg per 70 kg body weight to about 325 mg per 70 kg body weight being most preferred in the unit dosage form.

The nontoxic NMDA receptor antagonist must be present in the combined dosage form in an opioid euphoria-inhibiting amount. It would be recognized by one skilled in the art that this will relate to the particular opioid analgesic present and its euphoria-inducing capacity which, in turn, is believed to be related to its abuse potential. The amount of nontoxic NMDA receptor antagonist for combination with a specific opioid analgesic in a particular combined unit dosage form will depend upon the nature and amount of the opioid and its euphoria-inducing capacity and the nature of the nontoxic NMDA receptor antagonist and its ability to produce an opioid euphoria-inhibiting effect, as well as the particular formulation containing the active substances and the state and circumstances of the host being treated. As those skilled in the art will recognize, many factors that modify the action of the active substances herein will be taken into account by the treating physician such as the age, body weight, sex, diet and condition of the subject, the time of administration, the rate and route of administration, and so forth. Optimal dosages for a given set of conditions can be ascertained by those skilled in the art using conventional dosage determination tests. Table 1 below sets forth ranges for several specific opioid analgesics and a preferred nontoxic NMDA receptor antagonist, dextromethorphan.

The composition herein can be formulated as a solid, liquid, powder, elixir, injectable solution, etc. When formulated for oral delivery, the combination of drugs herein may be in the form of tablets, liquids, troches, lozenges, quick dissolve tablets, aqueous or oily suspensions, multiparticulate formulations including dispersible powders, granules, carrier spheroids or coated inert beads, emulsions, hard or soft capsules or syrups or elixirs, microparticles (e.g., microcapsules, microspheres and the like), buccal tablets, etc. The opioid analgesic and NMDA receptor antagonist can be employed in admixtures with conventional excipients, i.e., pharmaceutically acceptable organic or inorganic substances suitable for oral administration, known to those skilled in the art. Suitable pharmaceutically acceptable substances include but are not limited to water, salt solutions, alcohols, gum arabic, vegetable oils, benzyl alcohols, polyethylene glycols, gelate, carbohydrates such as lactose, amylose or starch, magnesium stearate, talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For oral administration, particularly suitable are tablets, dragees, liquids, drops, suppositories, or capsules, caplets and gelcaps. The compositions intended for oral use may be prepared according to any method known in the art. When prepared as tablets, the tablets may be uncoated or they may be coated by known techniques for elegance or to delay release of the active ingredients. Formulations for oral use may also be presented as hard gelatin capsules wherein the active ingredient is mixed with an inert diluent.

The dosage forms may further provide an immediate release of the opioid analgesic and the NMDA receptor antagonist. In certain preferred embodiments, the dosage forms provide a sustained release of the opioid analgesic, and provide the part or all of the dose of NMDA receptor antagonist in (i) immediate release form, (ii) sustained release form, or (iii) both immediate and sustained release form. Such embodiments may further comprise a portion of the opioid analgesic in immediate release form. Sustained release may be accomplished in accordance with formulations/methods of manufacture known to those skilled in the art of pharmaceutical formulation, e.g., via the incorporation of the opioid analgesic and NMDA receptor antagonist in a controlled release carrier; or via a controlled release coating of a carrier containing the opioid analgesic and NMDA receptor antagonist.

In one embodiment, the pharmaceutical dosage form comprises a sustained release carrier. Alternatively, a normal release carrier having a coating that controls the release of the drug may be used. Suitable base materials for controlled release carriers include combinations of higher aliphatic alcohols and acrylic resins.

Base compositions prepared from such higher aliphatic alcohols and acrylic resins provide sustained release of therapeutically active ingredients over a period of time from five hours and for as much as 24 hours after administration, generally oral administration, in humans or animals.

These bases can be prepared from any pharmaceutically acceptable higher aliphatic alcohol, the most preferred being fatty alcohols of 10-18 carbon atoms, particularly stearyl alcohol, cetyl alcohol, cetostearyl alcohol, lauryl alcohol, myristyl alcohol and mixtures thereof.

Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. These polymers can be synthesized, as indicated above, to be cationic, anionic or non-ionic, which then renders the polymers that would be pH dependent and consequently soluble in, or resistant to solutions over a wide range in pH.

In addition, suitable materials for inclusion in a controlled release carrier include:

(a) Hydrophilic polymers, such as gums, cellulose ethers, acrylic resins and protein derived materials. Of these polymers, the cellulose ethers, especially hydroxyalkylcelluloses and carboxyalkylcelluloses, are preferred. The dosage form may contain between 1% and 80% (by weight) of at least one hydrophilic or hydrophobic polymer.

(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred. The oral dosage form may contain up to 60% (by weight) of at least one digestible, long chain hydrocarbon.

(c) Polyalkylene glycols. The oral dosage form may contain up to 60% (by weight) of at least one polyalkylene glycol.

One particularly suitable carrier comprises at least one water soluble hydroxyalkyl cellulose, at least one C₁₂-C₃₆, preferably C₁₄-C₂₂, aliphatic alcohol and, optionally, at least one polyalkylene glycol.

The at least one hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present pharmaceutical dosage form will be determined, inter alia, by the precise rate of opioid analgesic release required. Preferably however, the oral dosage form contains between 1% and 45%, especially between 5% and 25% (by weight) of the at least one hydroxyalkyl cellulose.

While the at least one aliphatic alcohol may be, for example, lauryl alcohol, myristyl alcohol or stearyl alcohol, in particularly preferred embodiments the at least one aliphatic alcohol is cetyl alcohol or cetostearyl alcohol. The amount of the at least one aliphatic alcohol in the present dosage form will be determined, as above, by the precise rate of opioid analgesic release required. It will also depend on whether at least one polyalkylene glycol is present in or absent from the dosage form. In the absence of at least one polyalkylene glycol, the dosage form preferably contains between 20% and 50% (by weight) of the at least one aliphatic alcohol. When at least one polyalkylene glycol is present in the dosage form, then the combined weight of the at least one aliphatic alcohol and the at least one polyalkylene glycol preferably constitutes between 20% and 50% (by weight) of the total dosage.

In the present preferred dosage form, the ratio of, e.g., the at least one hydroxyalkyl cellulose or acrylic resin to the at least one aliphatic alcohol/polyalkylene glycol determines, to a considerable extent, the release rate of the opioid analgesic from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or polyethylene glycol, which is preferred. The number average molecular weight of the at least one polyalkylene glycol is preferred between 1000 and 15000 especially between 1500 and 12000.

Another suitable controlled release carrier would comprise an alkylcellulose (especially ethyl cellulose), a C₁₂ to C₃₆ aliphatic alcohol and, optionally, a polyalkylene glycol.

In addition to the above ingredients, a controlled release carrier may also contain suitable quantities of other materials, e.g. diluents, lubricants, binders, granulating aids, colorants, flavorants and glidants that are conventional in the pharmaceutical art.

As an alternative to a controlled release carrier, the present carrier may be a normal release carrier having a coat that controls the release of the drug. In particularly preferred embodiments of this aspect of the invention, the present dosage form comprises film coated spheroids containing active ingredient and a non-water soluble spheronising agent. The term spheroid is known in the pharmaceutical art and means a spherical granule having a diameter of between 0.5 mm and 2.5 mm especially between 0.5 mm and 2 mm.

The spheronising agent may be any pharmaceutically acceptable material that, together with the active ingredient, can be spheronised to form spheroids. Microcrystalline cellulose is preferred. According to a preferred aspect of the present invention, the film coated spheroids contain between 70% and 99% (by wt), especially between 80% and 95% (by wt), of the spheronising agent, especially microcrystalline cellulose.

In addition to the active ingredient and spheronising agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the opioid analgesic at a controlled rate in an aqueous medium. The film coat is chosen so as to achieve, in combination with the other ingredients, the in-vitro release rate outlined above (between 12.5% and 42.5% (by weight) release after 1 hour, etc.).

The film coat will generally include a water insoluble material such as: (a) a wax, either alone or in admixture with a fatty alcohol; (b) shellac or zein; (c) a water insoluble cellulose, especially ethyl cellulose; (d) a polymethacrylate.

Preferably, the film coat comprises a mixture of the water insoluble material and a water soluble material. The ratio of water insoluble to water soluble material is determined by, amongst other factors, the release rate required and the solubility characteristics of the materials selected.

The water soluble material may be, for example, polyvinylpyrrolidone or, which is preferred, a water soluble cellulose, especially hydroxypropylmethyl cellulose.

Suitable combinations of water insoluble and water soluble materials for the film coat include shellac and polyvinylpyrrolidone or, which is preferred, ethyl cellulose and hydroxypropylmethyl cellulose.

In another embodiment, in order to obtain a sustained-release of the opioid sufficient to provide an analgesic effect for the extended durations set forth in the present invention, the substrate comprising the therapeutically active agent may be coated with a sufficient amount of hydrophobic material to obtain a weight gain level from about 2 to about 30 percent, although the overcoat may be greater depending upon the physical properties of the particular opioid analgesic compound utilized and the desired release rate, among other things.

The solvent which is used for the hydrophobic material may be any pharmaceutically acceptable solvent, including water, methanol, ethanol, methylene chloride and mixtures thereof. It is preferable however, that the coatings be based upon aqueous dispersions of the hydrophobic material.

In certain preferred embodiments of the present invention, the hydrophobic polymer comprising the sustained-release coating is a pharmaceutically acceptable acrylic polymer, including but not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

In other preferred embodiments, the hydrophobic polymer which may be used for coating the substrates of the present invention is a hydrophobic cellulosic material such as ethylcellulose. Those skilled in the art will appreciate that other cellulosic polymers, including other alkyl cellulosic polymers, may be substituted for part or all of the ethylcellulose included in the hydrophobic polymer coatings of the present invention.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is especially preferred.

Examples of suitable plasticizers for the acrylic polymers of the present invention include citric acid esters such as triethyl citrate NF XVI, tributyl citrate, dibutyl phthalate, and possibly 1,2-propylene glycol, polyethylene glycols, propylene glycol, diethyl phthalate, castor oil, and triacetin, although it is possible that other water-insoluble plasticizers (such as acetylated monoglycerides, phthalate esters, castor oil, etc.) may be used. Triethyl citrate is especially preferred.

The sustained-release profile of the formulations of the invention can be altered, for example, by varying the thickness of the hydrophobic coating, changing the particular hydrophobic material used, or altering the relative amounts of, e.g., different acrylic resin lacquers, altering the manner in which the plasticizer is added (e.g., when the sustained-release coating is derived from an aqueous dispersion of hydrophobic polymer), by varying the amount of plasticizer relative to hydrophobic polymer, by the inclusion of additional ingredients or excipients, by altering the method of manufacture, etc.

Sustained-release spheroids or beads, coated with a therapeutically active agent are prepared, e.g. by dissolving the opioid analgesic in water and then spraying the solution onto a substrate using a Wurster insert. Optionally, additional ingredients are also added prior to coating the beads in order to assist the opioid analgesic binding to the substrates, and/or to color the solution, etc. For example, a product which includes hydroxypropyl methylcellulose, etc. with or without colorant may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic sustained-release coating. An example of a suitable barrier agent is one which comprises hydroxypropyl methylcellulose. However, any film-former known in the art may be used. It is preferred that the barrier agent does not affect the dissolution rate of the final product.

The coating solutions of the present invention may contain, in addition to the film-former, plasticizer, and solvent system (i.e., water), a colorant to provide elegance and product distinction. Color may be added to the solution of the therapeutically active agent instead, or in addition to the aqueous dispersion of hydrophobic polymer.

The plasticized aqueous dispersion of hydrophobic polymer may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic polymer to obtain a predetermined sustained-release of said therapeutically active agent when said coated substrate is exposed to aqueous solutions, e.g. gastric fluid, is preferably applied, taking into account the physically characteristics of the therapeutically active agent, the manner of incorporation of the plasticizer, etc. After coating with the hydrophobic polymer, a further overcoat of a film-former is optionally applied to the beads. This overcoat is provided, if at all, in order to substantially reduce agglomeration of the beads.

Next, the coated beads are cured in order to obtain a stabilized release rate of the therapeutically active agent.

In another embodiment, the pharmaceutical dosage form of the present invention is an aqueous suspension. Aqueous suspensions can contain the composition in admixture with pharmaceutically acceptable excipients such as suspending agents, e.g., sodium carboxymethyl cellulose, methylcellulose, hydroxypropylmethylcellulose, sodium alginate, polyvinylpyrrolidone, and natural gums such as gum tragacanth and gum acacia; dispersing or wetting agents such as naturally occurring phosphatide and lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monooleate. Such aqueous suspensions can also contain one or more preservatives, e.g., ethyl- or n-propyl-p-hydroxy benzoate, one or more coloring agents, one or more flavoring agents and one or more sweetening agents, such as sucrose, saccharin or sodium or calcium cyclamate.

Dispersible powders and granules suitable for preparation of an aqueous suspension by the addition of water provide the composition in admixture with a dispersing of wetting agent, suspending agents and one or more preservatives. Suitable dispersing or wetting agents and suspending agents are exemplified by those already mentioned above. Additional excipients, e.g., sweetening, flavoring and coloring agents, can also be present. Syrups and elixirs can be formulated with sweetening agents, for example glycerol, sorbitol or sucrose. Such formulations can also contain a demulcent, a preservative and flavoring and coloring agents.

Generally the amount of NMDA receptor antagonist used in the pharmaceutical dosage form the present invention will vary with the amount and type of opioid analgestic used. Listed below in Table 1 are some examples of the combined opioid analgestic and NMDA receptor antagonist that can be utilized in accordance with the present disclosure. It should be understood that any numerical value provided is approximate and should be construed to mean approximately or about that number.

TABLE 1
SOLID DOSAGE FORMS

| | OPIOID ANALGESIC, | NMDA RECEPTOR |
|---|---|---|
| EX- | mg per 70 kg body | ANTAGONIST, mg |
| AMPLE | weight per unit dose | per 70 kg body weight per unit dose |
| 1 | codeine, 5-360 | dextromethorphan HBr, 5-500 |
| 2 | dihydrocodeine, 2-200 | dextromethorphan HBr, 5-500 |
| 3 | hydrocodone, 2-400 | dextromethorphan HBr, 5-500 |
| 4 | hydromorphone, 4-64 | dextromethorphan HBr, 10-500 |
| 5 | morphine, 5-800 | dextromethorphan HBr, 10-500 |
| 6 | oxycodone, 5-400 | dextromethorphan HBr, 10-500 |
| 7 | oxymorphone, 2-100 | dextromethorphan HBr, 10-500 |
| 8 | traniadol, 25-200 | dextromethorphan HBr, 10-250 |
| 9 | propirani, 25-200 | dextromethorphan HBr, 5-500 |

It should be understood that various modifications may be made to the embodiments disclosed herein. Therefore, the above description should not be construed as limiting, but merely as exemplifications of preferred embodiments. For example, NMDA receptor antagonists other than dextromethorphan can be utilized in the pharmaceutical dosage form described herein. Those skilled in the art will envision other modifications within the scope and spirit of the claims appended hereto.

What is claimed is:

1. An abuse-resistant opioid-containing pharmaceutical dosage form which comprises an analgesically effective amount of opioid analgesic and an opioid euphoria-inhibiting amount of at least one nontoxic N-methyl-D-aspartate receptor antagonist, the dosage form being substantially free of opioid antagonist.

2. The dosage form of claim 1 wherein the opioid analgesic is at least one member selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

3. The dosage form of claim 1 wherein the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts.

4. The dosage form of claim 1 wherein the nontoxic NMDA receptor antagonist is at least one member selected from the group consisting of dextromethorphan, dextrorphan, memantine, amantidine, d-methadone and their pharmaceutically acceptable salts.

5. The dosage form of claim 3 wherein the nontoxic NMDA receptor antagonist is at least one member selected from the group consisting of dextromethorphan, dextrorphan, memantine, amantidine, d-methadone and their pharmaceutically acceptable salts.

6. The dosage form of claim 1 which is a solid dosage form.

7. The dosage form of claim 6 wherein the opioid analgesic is in a controlled release carrier.

8. The dosage form of claim 7 wherein the controlled release carrier comprises a base material selected from the group consisting of a hydrophilic polymer, a hydrophobic polymer, a long chain hydrocarbon, a polyalkylene glycol, higher aliphatic alcohols, acrylic resins, and mixtures thereof.

9. The dosage form of claim 7 wherein the controlled release carrier comprises a base material having a coating that controls the release of the opioid analgesic.

10. The dosage form of claim 1 which is a liquid dosage form.

11. The dosage form of claim 10 which is an injectable dosage form.

12. The dosage form of claim 1 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

13. The dosage form of claim 1 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

14. The dosage form of claim 1 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

15. The dosage form of claim 6 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

16. The dosage form of claim 6 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

17. The dosage form of claim 6 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

18. The dosage form of claim 11 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

19. The dosage form of claim 11 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

20. The dosage form of claim 11 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

21. An abuse-resistant opioid-containing pharmaceutical solid dosage form which comprises an analgesically effective amount of at least one opioid analgesic selected from the group consisting of codeine, dihydrocodeine, hydrocodone, hydromorphone, levorphanol, meperidine, methadone, morphine, oxycodone, oxymorphone, propoxyphene, tramadol and their pharmaceutically acceptable salts and an opioid euphoria-inhibiting amount of dextromethorphan, the dosage form being substantially free of opioid antagonist.

22. The dosage form of claim 21 wherein the opioid analgesic is in a controlled release carrier.

23. The dosage form of claim 22 wherein the controlled release carrier comprises a base material selected from the group consisting of a hydrophilic polymer, a hydrophobic polymer, a long chain hydrocarbon, a polyalkylene glycol, higher aliphatic alcohols, acrylic resins, and mixtures thereof.

24. The dosage form of claim 21 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

25. The dosage form of claim 21 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

26. The dosage form of claim 22 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

27. The dosage form of claim 22 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

28. The dosage form of claim 23 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

29. The dosage form of claim 23 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

30. An abuse-resistant opioid-containing pharmaceutical liquid dosage form which comprises an analgesically effective amount of at least one opioid analgesic selected from the group consisting of codeine, dihydrocodeine, hydrocodone, hydromorphone, levorphanol, meperidine, methadone, morphine, oxycodone, oxymorphone, propoxyphene, tramadol and their pharmaceutically acceptable salts and an opioid euphoria-inhibiting amount of dextromethorphan, the dosage form being substantially free of opioid antagonist.

31. The dosage form of claim 30 wherein the dosage form is an injectable dosage form.

32. The dosage form of claim 31 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

33. The dosage form of claim 31 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

34. An opioid-containing pharmaceutical dosage form which is resistant to abuse by intranasal administration which comprises an analgesically effective amount of opioid analgesic substantially free of opioid antagonist and an intranasal mucosa-irritating amount of at least one nontoxic N-methyl-D-aspartate receptor antagonist.

35. The dosage form of claim 34 wherein the opioid analgesic is at least one member selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

36. The dosage form of claim 34 wherein the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts.

37. The dosage form of claim 34 wherein the nontoxic NMDA receptor antagonist is at least one member selected from the group consisting of dextromethorphan, dextrorphan, memantine, amantidine, d-methadone and their pharmaceutically acceptable salts.

38. The dosage form of claim 36 wherein the nontoxic NMDA receptor antagonist is at least one member selected from the group consisting of dextromethorphan, dextrorphan, memantine, amantidine, d-methadone and their pharmaceutically acceptable salts.

39. The dosage form of claim 34 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

40. The dosage form of claim 34 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

41. The dosage form of claim 34 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.
